(19) United States
(12) Patent Application Publication
(10) Pub. No.: US 2009/0125035 A1
(43) Pub. Date: May 14, 2009

(54) Surgical Cutting Instrument for Breast Surgery

(76) Inventor: John S. Kennedy, Stone Mountain, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 11/939,587

(22) Filed: Nov. 14, 2007

(51) Int. Cl. A61B 17/3205

(52) U.S. Cl. 606/110; 606/171

(57) ABSTRACT

A cutter (14) is telescopically received in a cannula (12). A detection device (54) may be inserted through the open-ended cutter and cannula to properly place the instrument in alignment with the tumor to be excised. The cutter and cannula are pressed into the tissue of the patient with the expectation that the circular core (60) of breast tissue formed by the cutter will have clear margins about the tumor. If the tumor extends too close to the circular incision, the cannula may be rotated so that its sidewall opening (26) faces the side of the remaining tissue to be excised and the surgeon can pull the remaining tissue through the sidewall and excise it, thereby avoiding a separate and delayed surgical procedure.

### FIELD OF THE INVENTION

This invention concerns a cutting instrument used by a surgeon during the process of extracting malignant or non-malignant tumors or similar tissues from various parts of the body, particularly for use in lumpectomy, extracting cancerous tumors from the breast.

### BACKGROUND OF THE INVENTION

When conducting breast surgery for the removal of tumors, the usual procedure is to locate the position and depth of the tumor by use of palpation, ultrasound, mammogram, or other detection devices. With this information, the surgeon typically makes a shallow incision in the skin so as to pull away the skin from the underlying tissue and then proceeds with surgery through the opening in the skin and into the fat and breast tissue to reach the tumor and excise it.

In recent years, various surgical aids have been developed for reaching and excising tumors. For example, a garrote wire has been used that has a loop that can be inserted over and beneath the tumor. The garrote loop is heated to a temperature sufficient for searing the tissue and the loop is drawn tightly beneath the tumor so as to release the tumor from the rest of the breast. This enables the surgeon to lift the tissue that contains the tumor from the patient. Other improvements include the use of cylindrically shaped cutters that cut a core of tissue from the breast, with the core surrounding the tumor and then excising the core with the tumor contained in the core.

Breast conserving surgery is considered the most desirable surgical option for the majority of women with breast cancer and has become the standard of care for most women with breast cancer. Desirably, the incision should be made about the tumor with a clear negative margin of tissue about the tumor so as to make sure that the entire tumor is excised.

Typically, when the tissue has been removed from the body, a pathologist examines the tissue to determine the nature of the cancerous growth and particularly to determine if the tumor extends beyond the tissue removed from the patient, or if the clear negative margin of tissue about the tumor is not adequate. If a portion of the tumor has been left in the patient or if a clear margin about the tumor is not adequate, a reexcision must be made.

One option for achieving a clear margin would be to remove a much larger amount of tissue about the tumor, however this compromises the cosmetic outcome of a procedure which is intended to conserve the contour of the breast. In addition, excision of more tissue is likely to extend the time for healing and recovery. The risk of bleeding and infection is likely increased as well. Another option for achieving a clear margin is to simply wait for the final pathology report, typically available a few days later. If the tumor has not been completely removed, or if the margin of tissue about the tumor is not adequate, a reexcision can be done. A reexcision would usually be made about two weeks after the initial surgery. The reexcision rates in the published literature range between 15% up to more than 50% of the initial operations. Reexcision usually is demoralizing and a physical ordeal for the patient, let alone the costs, added recovery time, and added risks of a second procedure.

Accordingly, it would be desirable for both the patient and the surgeon to use a device and a process for more reliably and expediently removing tumors from the body, with the device and with the procedure forming a small opening in the breast tissue for excising the tumor with a comparatively small amount of surrounding normal breast tissue, and providing the ability to reexcise about the initial excision cavity to form clear negative margins of tissue about the side of the tumor before termination of the overall initial procedure.

### SUMMARY OF THE INVENTION

Briefly, this invention concerns the removal of fibroid tumors and the like from breasts and possibly from other portions of the human body. A surgical cutting instrument for excising tumors from a body is provided. It includes a cylindrical open-ended cannula that includes a cylindrical sidewall and at least one opening in the sidewall. A cutter is telescopically received in the cannula, and the cutter includes a cutting edge for extending beyond the distal end of the cannula. The cutter is used for cutting the tissue and forming an opening for receiving the cannula as the cannula and cutter are inserted into the body. The cutter may be rotated to form the incision.

In one embodiment of the invention, direction indicia are applied to the proximal end of the cannula for indication of the radial direction at which a tumor or narrow margin about a tumor of a patient extends outside the incision. For example, the direction indicia may include clock markings such as 3, 6, 9 and 12, about the rim at the proximal end to the cannula, compass markings such as 0, 90, 180, and 270, or other similar direction indicia.

Once the incision has been made, the cutter may be removed from the cannula and from about the core of tissue that has been formed by the cutter, leaving the cannula in place about the core of tissue.

A garrote wire may be carried with the instrument into the incision. The garrote wire has a loop that extends about the inside of the cannula so that the loop surrounds the core of tissue formed by the cutter. The garrote wire has a tail extending from the loop to outside of the cannula, up to the entrance of the incision, where it is accessible to the surgeon. The tail of the garrote wire may be pulled by the surgeon so as to close the loop and cut through the inner portion of the core cut by the cutter. This tends to cut a transection of tissue transverse to the core cut by the cutter to separate the core completely from the breast so that the surgeon may pull the core through the cannula.

Another feature of the invention may be the use of an ultrasound instrument with the cannula and cutter. The cannula and cutter both may be open-ended and sized and shaped for receiving an ultrasound instrument or some other detection device, such that the detection device is positioned within the cannula and the cutter for reading the position of the tumor. Typically, the detection device, the cannula and the cutter are placed on the breast of the patient so that the detection device may accurately locate the tumor and therefore accurately position the cannula and cutter in alignment with the tumor.

Once the cannula and cutter are properly located, the detection device may be removed during the remainder of the surgical procedure, or may remain in place in the cannula to continuously check the location of the tumor as the incision is formed.

The cannula may be formed with an opening in its sidewall, with the opening extending between approximately 70° to 100° about the sidewall of the cannula. After the initial incision has been made with the instrument and the tumor excised, the pathologist might discover that a clear negative margin was not formed at one side of the tumor. Upon receiving the pathologist's report, the surgeon may rotate the cannula about its longitudinal axis so as to have its sidewall opening register with the tissue where more tissue may be removed to form the clear margin, and the surgeon may excise more tissue to form the desired margin.

The invention may also include the method of excising tumors from the body by advancing the cannula and the cutter in telescoped relationship into the tissue of the body about a tumor to a depth that surrounds a core of tissue that contains the tumor. The surgeon may rotate the cannula to view through the opening of the cannula into the tissue facing the cannula and excise the tissue in registration with the opening of the cannula.

The method of excising the tumors may also include the use of the garrote wire, by positioning the garrote wire loop about the core of tissue and tightening the garrote wire so as to cut through the base of the core of tissue.

Other procedures and devices are set forth in more detail hereinafter and are described in more detail in the claims appended hereto.

### BRIEF DESCRIPTION OF THE DRAWINGS

FIG. 1 is a perspective view of the surgical cutting instrument without the detection device, including both the cannula and cutter in telescoped relationship, showing the top and side surfaces thereof.

FIG. 2 is an expanded perspective view of the surgical cutting instrument and a core of tissue cut from a patient.

FIG. 3 is a perspective view of the surgical instrument, showing how a mammogram instrument may be inserted through the open-ended instrument for the purpose of locating the tumor and for positioning the surgical instrument in registration with the tumor.

FIG. 4 is a plan view of the cannula after it has been inserted in the tissue of the patient, showing the tumor surrounded by the cannula.

FIG. 5 is a plan view of the cannula, similar to FIG. 4, showing how the cannula can be rotated so that its sidewall opening faces in the direction where more tissue is to be excised to form a clear margin about the tumor.

FIG. 6 is a plan view, similar to FIGS. 4 and 5, but showing how the surgeon uses an instrument for grasping the adjacent tissue and pulling it into the confines of the cannula where it may be expediently excised by the surgeon.

### DETAILED DESCRIPTION

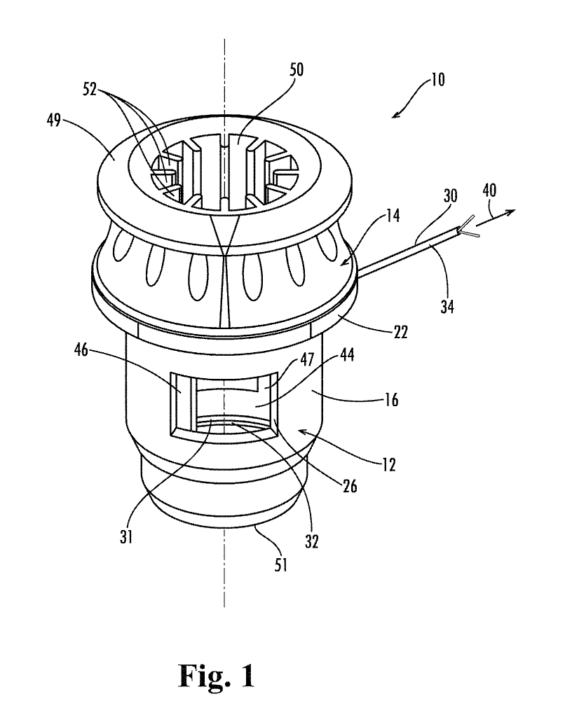

Referring now in more detail to FIG. 1, the surgical instrument 10 includes a cannula 12 and a cutter 14. The cannula and cutter both may be cylindrical and the cutter is telescopically received through the cannula. The cannula is open-ended and the cutter may be open ended.

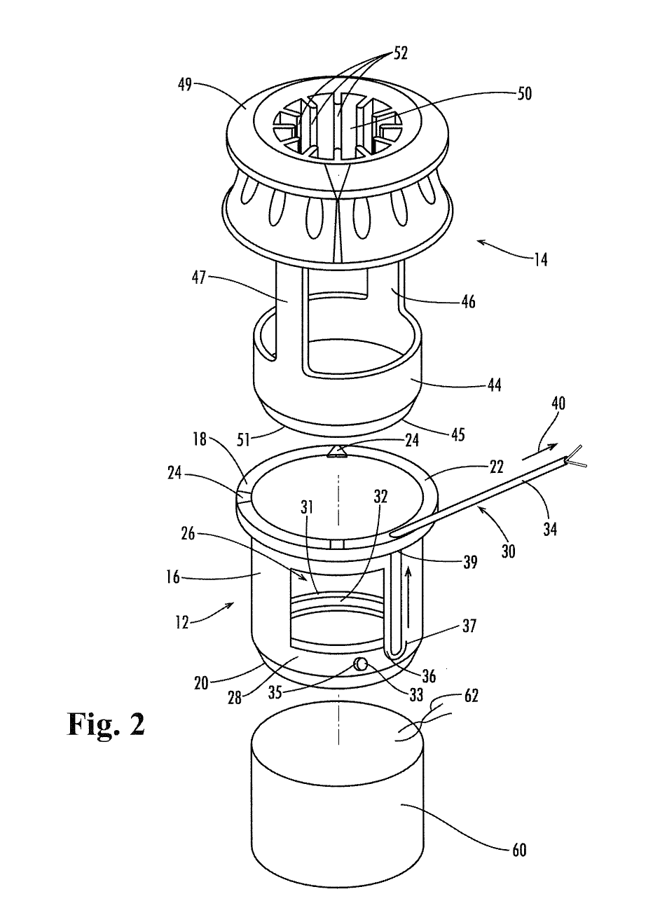

As shown in FIG. 2, the cannula 12 includes a cylindrical sidewall 16, a proximal end 18, and a distal end 20. The proximal end 18 includes a radially extending rim 22, and direction indicia may be imposed on the rim. For example, the direction indicia could include clock numbers such as 12, 3, 6 and 9; or compass numbers such as 0, 90, 180, and 270. Other direction indicia may be employed if desired.

The cannula includes at least one side opening such as sidewall opening 26 that is formed in the cylindrical sidewall 16. The sidewall opening 26 is generally rectangular and may occupy approximately 90° of the circular surface of the cylindrical sidewall 16. The height of the sidewall opening 26 may be 3 centimeters. The overall height of the cannula may be 5 centimeters. Other dimensions may be used as desired. The sidewall opening 26 may be of other shapes, such as oval or circular, as desired. The 12 o'clock position of the cannula 12 is aligned with the central portion of the sidewall opening 26 of the cannula.

The surface of the cannula at its distal end 20 is beveled inwardly so as to reduce the resistance applied against the tissue during the insertion of the surgical instrument into the tissue of the patient.

A garrote wire 30 may be carried by the cannula. The garrote wire 30 includes an internal loop 32 that is received in a complementary shaped groove 31 formed on the inside surface of the cannula cylindrical sidewall, as shown in FIGS. 1 and 2. The loop of the garrote wire is positioned between the facing surfaces of the cylindrical cutter and the cannula. The distal end 33 of the garrote wire may be attached to the cannula as by forming an enlarged portion of the garrote wire that extends through and frictionally engages the side port 35 of the cannula. The garrote tail 34 extends from inside the cannula through another port 37, then extends upwardly to the rim 22 through rim port 39, and from there outwardly so that it may be grasped by the surgeon or connected to another surgical implement. The presence of the cutter within the cannula prevents the loop of the garrote wire from closing. Typically, the garrote wire includes an electrically conductive wire with insulation about its tail and little or no insulation about its loop, and an electrical charge is applied to the wire, causing the loop to become hot for cutting through the tissue at the base of the core of tissue inside the cannula.

When the cutter 14 is withdrawn from the cannula 12, the tail 34 of the garrote wire 30 may be pulled in the direction as indicated by arrow 40 so as to contract the garrote loop 32, causing the garrote loop to become reduced in diameter and to cut through the tissue that is present within the cannula.

Cutter 14 includes a cutter ring 44, a pair of opposed support legs 46 and 47 that extend away from the cutter ring, and an enlarged hand grip 49 attached to the distal ends of the opposed support legs 46 and 47. The distal circular edge 51 of the cutter ring 44 is sharpened for cutting through the tissue of the patient. This arrangement allows the cutter 14 to be inserted telescopically into the cannula 12 with the hand grip 49 moved into engagement with the rim 22 of the cannula 12. The surgeon may rotate the hand grip of the cutter 14, causing the cutter ring 44 to cut through the tissue of the breast and to progressively enter the breast.

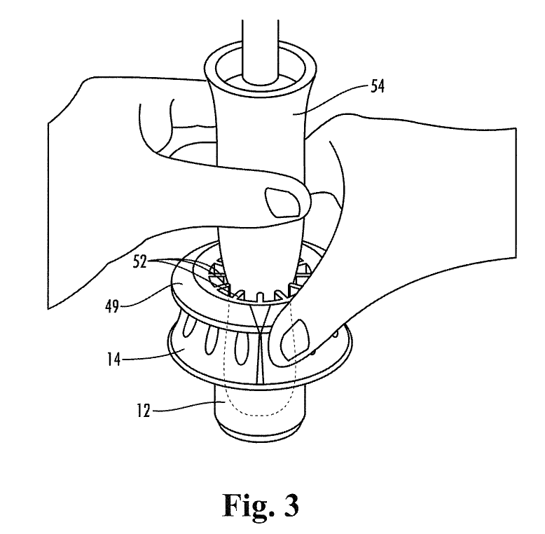

Both the cannula 12 and the cutter 14 may be open-ended. The cutter may be cylindrical and open-ended and may include a longitudinal opening 50 that extends through the hand grip 49. An array of support fins 52 extend radially inwardly of the external opening 50 of the cylindrical cutter. As shown in FIG. 3, the fins 52 of the cylindrical cutter may be used to guide and support a detection device 54, such as an ultrasound detector as the detection device is passed through the open-ended instrument.

The dimensions of the instrument are such that the cutting edge 45 of the cutter protrudes a predetermined distance beyond the distal end 20 of the cannula 12. This makes the cutting edge available for forming an incision in the tissue of the patient. The length of the legs 46 and 47 and the depth of the cutter ring 44 from the overhang of the hand grip 49 function to accurately position the cutting edge 45 at the predetermined distance beyond the distal end 20 of the cannula for forming the proper cut through the tissue of the patient.

FIG. 2 illustrates the cutter 14 suspended above the cannula 12 and shows the core 60 of tissue below the cannula that is excised from the patient. The instrument yields a more standardized cylindrical core 60 that includes a smoother contour that may decrease the likelihood of false margins due to the pathologist's ink extending into crevasses in the specimen.

Given the more consistent cylindrical shape of the specimen, orientation of the specimen can be more precise, and marking the specimen in accordance with the directional markings on the cannula may be more accurately perceived by the pathologist. The instrument more consistently excises a cylindrical core of tissue of a size that corresponds to the size and shape of the instrument. Such a standardized specimen provides additional information to the pathologist regarding orientation of the specimen and allows for more specific assessment of any marginal involvement. In turn, the surgeon can more accurately excise additional tissue as may be needed to achieve clear margins at the first procedure, or when needed by reexcision.

Another benefit of the instrument is that it yields a cavity in the tissue of the breast that is approximately cylindrical and of a standard size. This may improve cosmetic results and also the cavity may be used for partial breast irradiation using intra-cavity balloon brachytherapy as an alternative to external beam therapy, potentially reducing the treatment time of the therapy. Optimal use of the intra-cavity technique requires good formation of the balloon to the lumpectomy cavity. The more standardized cavity dimensions may make this procedure more reliable.

When the surgeon has used the detection device 54 as shown in FIG. 3 to properly position the cannula 12 and cylindrical cutter 14, the surgeon typically will remove the detection device and press the cannula and cylindrical cutter downwardly into the tissue of the patient so that the breadth of the cannula and cutter will surround the tumor. Preferably, the cannula and cutter chosen by the surgeon are of a sufficient breadth and length to form a negative margin about the tumor so that the surgeon and pathologist will be satisfied that the entire tumor has been surrounded and cut from the patient.

The incision formed by the cylindrical cutter 14 might take the shape of the core 60 shown in FIG. 2, which usually is somewhat cylindrically shaped.

Typically, the surgeon will mark the core 60 by forming a stitch 62 in the tissue of the core or by some other marking means at a predetermined position on the core 60, such as at the 12 o'clock position on the proximal surface of the core. This may be done before the core is excised.

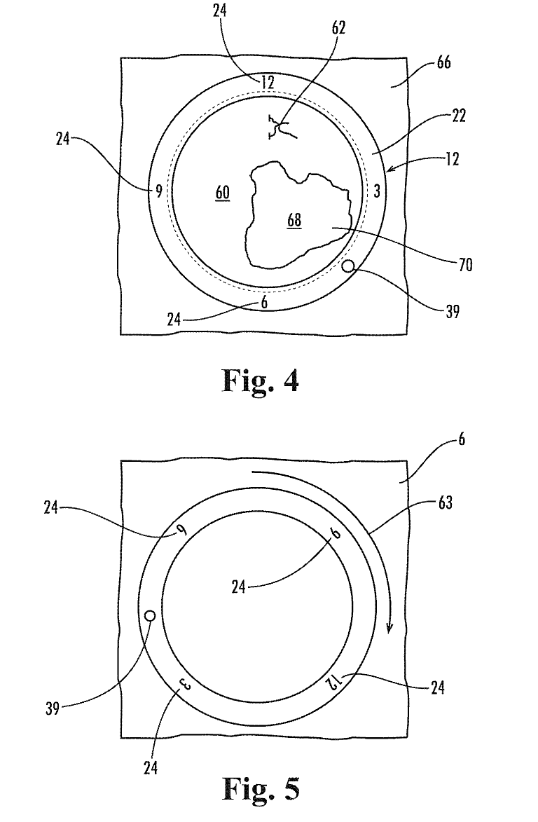

For example, FIG. 4 shows the cannula 12 and its rim 22 after the cutter and cannula have been inserted into the tissue 66 of the patient and with the cutter having been removed therefrom, leaving only the cannula in place. This forms the core of tissue 60.

Once the circular incision has been made, the core must be separated from the tissue at the distal end of the core. In order to separate the core 60 from the internal tissue at the distal end of the core, the garrote wire 30 may be actuated and pulled in the direction indicated by arrow 40, by pulling the tail of the garrote wire so that the loop 32 of the garrote wire contracts and squeezes into the base of the core of the tissue 60, tending to cut through the tissue. The garrote wire may be electrified so as to become hot and the heat of the garrote wire tends to expedite the cutting function as the garrote loop contracts through the tissue, forming the bottom surface of the core.

While a garrote wire may be used as described, other means may be used to separate the core from the body.

When the core 60 (FIG. 2) has been removed from the cannula, it is likely that the core of tissue will be submitted to a pathologist for evaluation. In the example of FIG. 4, the pathologist is likely to detect that the tumor 68 is not perfectly centered in the core 60, leaving only a small margin of tissue at 70 about the tumor.

The pathologist will inform the surgeon that the negative margin about the tumor is not satisfactory and will inform the surgeon of the location of the inadequate margin. The pathologist will understand that the stitch 62 was formed at the 12 o'clock position of the core of tissue, and the pathologist can estimate the position of the inadequate margin. For example, FIG. 4 shows that the inadequate negative margin of tissue about the tumor is between the 3 o'clock and 4 o'clock positions when measured clockwise from the stitch 62.

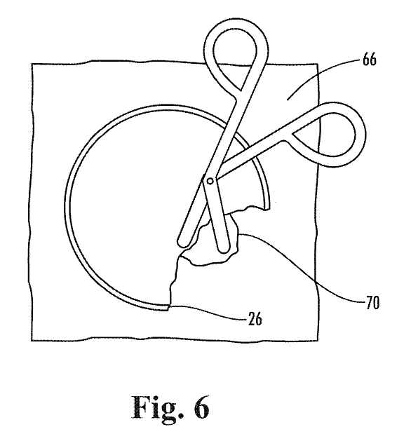

With the above information, the surgeon will then be able to rotate the cannula in the breast of the patient as shown by arrow 63 until the 12 o'clock indicia faces between the 3 and 4 o'clock positions, as shown in FIG. 5. Since the 12 o'clock position on the rim of the cannula is aligned with the sidewall opening 26 of the cannula, the surgeon will then have the sidewall opening 26 in registration with the position of the inadequate negative margin 70 that was adjacent the tumor. The surgeon is then able to pull the inadequate margin of tissue through the sidewall opening 26 as shown in FIG. 6 with an appropriate instrument and then surgically remove the portion of the tissue 70 at the inadequate margin.

Once this procedure has been accomplished, the cannula can be withdrawn from the incision and the tissue surrounding the cannula tends to collapse inwardly to close the wound.

It should be noted that the presence of the cannula in the incision tends to retard bleeding into the opening of the tissue.

An advantage provided by the invention is that one surgical procedure can be used for the expected incision about the tumor with clear negative margins about the tumor so that no reexcising of the portions of the tumor will be required. However, should there be an inadequate margin about the tumor, or if the tumor is discovered to extend out of the core, the reexcision as described above can be performed without having to perform additional surgery through the surface tissue of the patient. This tends to avoid the additional trauma that may be experienced by the patient for a follow-up surgical procedure.

The cannula and the cutter may be made of hard plastic, metal or other substances suitable for the intended use of the instrument. If desired, parts of the device may be made of re-useable materials such as stainless steel.

The instrument may be made in different sizes as may be desired. It has been found for most surgeries for breast tumors, the preferred dimensions are approximately five centimeters diameter of the cannula, approximately five centimeters in height of the cannula, with the sidewall opening of the cannula being about 2.5 centimeters high and about 90° in circumferential dimension.

The external dimensions of the cutter should match the internal dimensions of the cannula, with the circular cutting edge of the cutter protruding from the distal end of the cannula when the cutter is fully telescoped into the cannula. The cutter may be cylindrically shaped to surround the core of tissue to be removed from the breast of the patient.

While the invention has been disclosed for use in breast surgery, it may be used for other surgical purposes.

Although preferred embodiments of the invention have been disclosed in detail herein, it will be obvious to those skilled in the art that variations and modifications of the disclosed embodiments can be made without departing from the spirit and scope of the invention as set forth in the following claims.

1. A surgical cutting instrument for excising tumors from a body, comprising:
a cylindrical open ended cannula including a cylindrical sidewall, a proximal end and a distal end, and at least one opening in said sidewall,
a cutter telescopically received in said cannula, said cutter including a circular cutting edge for extending beyond said distal end of said cannula for cutting the tissue and forming an opening for receiving the cannula as the cannula and cutter are inserted in the body,
direction indicia applied to said proximal end of said cannula for indication of the direction at which a tumor of the patient has spread outside the incision,
such that the cutter is removable from the cannula and the side of the tissue from which the excision was removed from the body where reexcision is to be performed may be exposed by rotation of the cannula until the opening of the cannula faces the area for reexcision.

2. The surgical cutting instrument of claim 1, wherein a garrote wire extends about the interior of said cannula for severing the tissue below the tumor.

3. The surgical cutting instrument of claim 2, wherein said garrote wire includes a loop that extends about the interior of said cannula and a tail extending from said loop outside said cannula.

4. The surgical cutting instrument of claim 1, wherein said cutter is cylindrical and open ended and sized and shaped for receiving an ultrasound instrument, such that the ultra sound instrument is positioned within said cannula and said cutter for reading the position of the tumor.

5. The surgical cutting instrument of claim 1, wherein said cutter is open ended and defines a longitudinal opening there through that includes an array of radial fins extending into the opening for supporting an ultrasound instrument.

6. The surgical cutting instrument of claim 1, wherein said opening of said cannula extends between 70° and 100° about said cannula.

7. The surgical cutting instrument of claim 1, wherein said cutter includes a hand grasping rim, at least two support legs extending from said rim, and a circular cutter supported by said supporting legs, with openings formed between said support legs.

8. A method of excising tumors from a body, comprising:
advancing a cannula and a cylindrical cutter in telescoped relationship with the cannula into the tissue of the body and about a tumor to a depth that surrounds the tumor with the cylindrical cutter protruding from the cannula,
cutting with the cutter the tissue of the body as the cannula and the cutter are advanced into the tissue of the body and forming a core of tissue of the body surrounded by the cutter and cannula,
withdrawing the cutter from within the cannula,
extracting the core from the body,
rotating the cannula to view through an opening in the cannula the tissue facing the cannula, and
excising tissue in registration with the opening in the cannula.

9. The method of claim 8, wherein the step of extracting the core from the body comprises tightening a garrote about the core.

10. The method of claim 8, and further including marking the core at a position on the core that corresponds to a mark on the cannula.

11. A surgical cutting instrument for excising a tumor from a body, comprising:
an open ended cannula including a cylindrical sidewall, a proximal end and a distal end,
a cutter received in said cannula,
said cutter including a cutting edge configured for extending beyond the distal end of said cannula for cutting into and forming a core of tissue in the body that contains the tumor within the cannula and within the cutter, and
at least one opening in said sidewall of said cannula for movement in response to rotating the cannula in the cavity of tissue to face a part of the tissue outside the cannula.

12. The surgical cutting instrument for excising tumors from a body of claim 11, and further comprising a garrote wire extending about the interior of said cannula for separating the core of tissue containing the tumor from the body.

13. The surgical cutting instrument for excising tumors from a body of claim 11, and further including direction indicia applied to said proximal end of said cannula for indication of the direction where the negative margin of tissue about the tumor in the core of tissue is not adequate.
